(19) United States
(12) Patent Application Publication
(10) Pub. No.: US 2016/0287201 A1
(43) Pub. Date: Oct. 6, 2016

(54) ONE OR MORE TWO DIMENSIONAL (2D) PLANNING PROJECTION IMAGES BASED ON THREE DIMENSIONAL (3D) PRE-SCAN IMAGE DATA

(71) Applicant: KONINKLIJKE PHILIPS N.V., Eindhoven (NL)

(72) Inventors: Martin BERGTHOLDT, HAMBURG (DE); Rafael WIEMKER, KISDORF (DE); Cristian LORENZ, HAMBURG (DE); Tobias KLINDER, UELZEN (DE)

(73) Assignee: Koninklijke Philips N.V.

(21) Appl. No.: 15/035,565

(22) PCT Filed: Oct. 31, 2014
(86) PCT No.: PCT/IB2014/065729

§ 371 Date: May 10, 2016

Related U.S. Application Data
(60) Provisional application No. 61/905,550, filed on Nov. 18, 2013.

(51) Int. Cl. A61B 6/00; A61B 6/03

(52) U.S. Cl. CPC A61B 6/5223; A61B 6/032; A61B 6/488; A61B 6/4435

(57) ABSTRACT

A method includes obtaining 3D pre-scan image data generated from a scan of a subject. The 3D pre-scan image data includes voxels that represent a tissue of interest. The method further includes generating a 2D planning projection image showing the tissue of interest based on the 3D pre-scan image data. A system includes a 2D planning projection image from 3D pre-scan image data generator (218). The 2D planning projection image from 3D pre-scan image data generator obtains 3D pre-scan image data generated from a scan of a subject. The 3D pre-scan image data includes voxels that represent a tissue of interest. The 2D planning projection image from 3D pre-scan image data generator further generates a 2D planning projection image showing the tissue of interest based on the 3D pre-scan image data.

The following generally relates to imaging and more particularly to generating one or more 2D planning projection images based on 3D pre-scan image data, and is described with particular application to computed tomography (CT). However, the following is also amenable to other imaging modalities.

A CT scanner includes an x-ray tube that emits radiation that traverses an examination region and an object therein. A detector array located opposite the examination region across from the x-ray tube detects radiation that traverses the examination region and the object therein and generates projection data indicative of the examination region and the object therein. A reconstructor processes the projection data and reconstructs volumetric image data indicative of the examination region and the object therein.

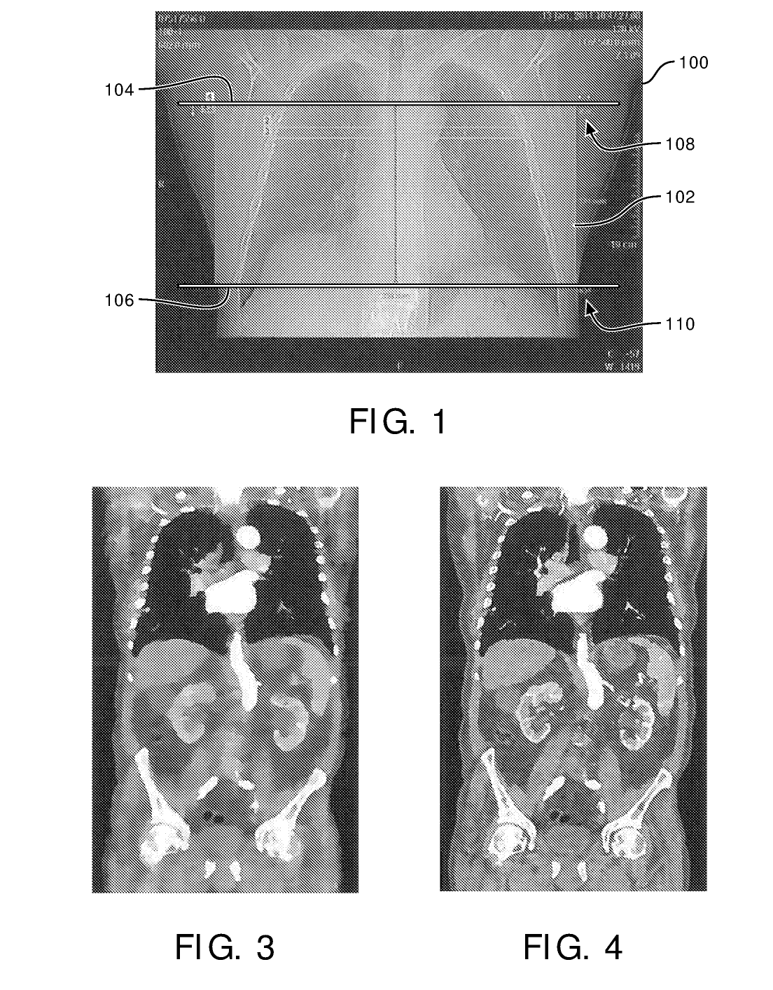

Planning a volume scan has included performing a two-dimensional (2D) pre-scan, which produces a 2D projection image. FIG. 1 shows an example of a projection image 100. With a 2D pre-scan, the location of the support supporting the patient, with respect to the image plane, is known. As such, the location of the anatomy in the 2D projection scan, with respect to the support and the image plane is known, if the patient does not move on the support.

The user defines a bounding box 102, which defines a field of view, which is the region that will be scanned during the volume scan. The bounding box 102 identifies a start scan position 104 and an end scan position 106. In the illustrated example, start and end 108 and 110 support positions are shown next to the start scan position 104 in the 2D projection image. Once a plan is created, the plan is used by the imaging system to perform a volume scan from the start scan position 104 to the end scan position 106.

With a 2D pre-scan, the 3D anatomical information is projected on a 2D display. As such, a pixel in the 2D projection image has an intensity value that represents a summation of the individual intensity values of the individual voxels corresponding to the pixel. As a result, anatomy in front of and/or behind tissue of interest may obscure the boundaries of the tissue of interest. A margin can be added to bounding box 102 to ensure adequate coverage.

A similar approach can be used with three-dimensional (3D) pre-scans. However, with a 3D pre-scan, the user scrolls through the slices of the pre-scan volume and creates the bounding box on one of the slices. This allows the user to find a slice in which less tissue obscures the boundaries of the tissue of interest, which facilitates optimizing the size of the bounding box to the tissue of interest and dose. Unfortunately, this approach consumes more user time since the user scrolls through the pre-scan volume.

The 3D pre-scan image data also allows the user to select one or more planning directions. For example, the coronal plane can be shown to provide a view similar to that shown in FIG. 1. However, the 3D pre-scan image data can be reformatted to show the sagittal plane, the axial plane, and/or an oblique plane. For each plane, the approach discussed in the previous paragraph would be used to locate a slice of interest and create the bounding box. This would, of course, consume even more of the user's time.

Aspects described herein address the above-referenced problems and others.

The following describes an approach for generating one or more 2D volume scan planning images from 3D pre-scan image data. This includes, in one instance, locating tissue(s) of interest in the volume of the 3D pre-scan image data and then selecting a sub-volume of the 3D pre-scan image data that includes the located tissue(s) of interest. The one or more 2D volume scan planning images are generated based on the sub-volume. The one or more 2D volume scan planning images may have improved image quality with respect to identifying the perimeter and/or boundaries associated with the tissue of interest, relative to a configuration in which the entire 3D pre-scan image data is used to generate the one or more 2D volume scan planning images where structure in front of and/or behind the tissue of interest visually obscures the perimeter and/or boundaries of the tissue of interest.

In one aspect, a method includes obtaining 3D pre-scan image data generated from a scan of a subject. The 3D pre-scan image data includes voxels that represent a tissue of interest. The method further includes generating a 2D planning projection image showing the tissue of interest based on the 3D pre-scan image data.

In another aspect, an imaging system includes a 2D planning projection image from 3D pre-scan image data generator. The 2D planning projection image from 3D pre-scan image data generator obtains 3D pre-scan image data generated from a scan of a subject. The 3D pre-scan image data includes voxels that represent a tissue of interest. The 2D planning projection image from 3D pre-scan image data generator further generates a 2D planning projection image showing the tissue of interest based on the 3D pre-scan image data.

In another aspect, computer readable instructions are encoded on computer readable storage medium, which, when executed by a processor of a computing system, cause the processor to: obtain 3D pre-scan image data generated from a scan of a subject, wherein the 3D pre-scan image data includes voxels that represent a tissue of interest, detect the tissue of interest in the 3D pre-scan image data, generate at least one region of interest in the 3D pre-scan image data, select a sub-volume of the 3D pre-scan image data based on the at least one region of interest, wherein the sub-volume bounds the region of interest, generate at least one 2D planning projection image for the tissue of interest based on the sub-volume of the 3D pre-scan and a view direction, plan a volume scan for the tissue of interest based on the 2D planning project image, and perform a scan of the subject based on the volume scan.

The invention may take form in various components and arrangements of components, and in various steps and arrangements of steps. The drawings are only for purposes of illustrating the preferred embodiments and are not to be construed as limiting the invention.

FIG. 1 illustrates a 2D projection image.

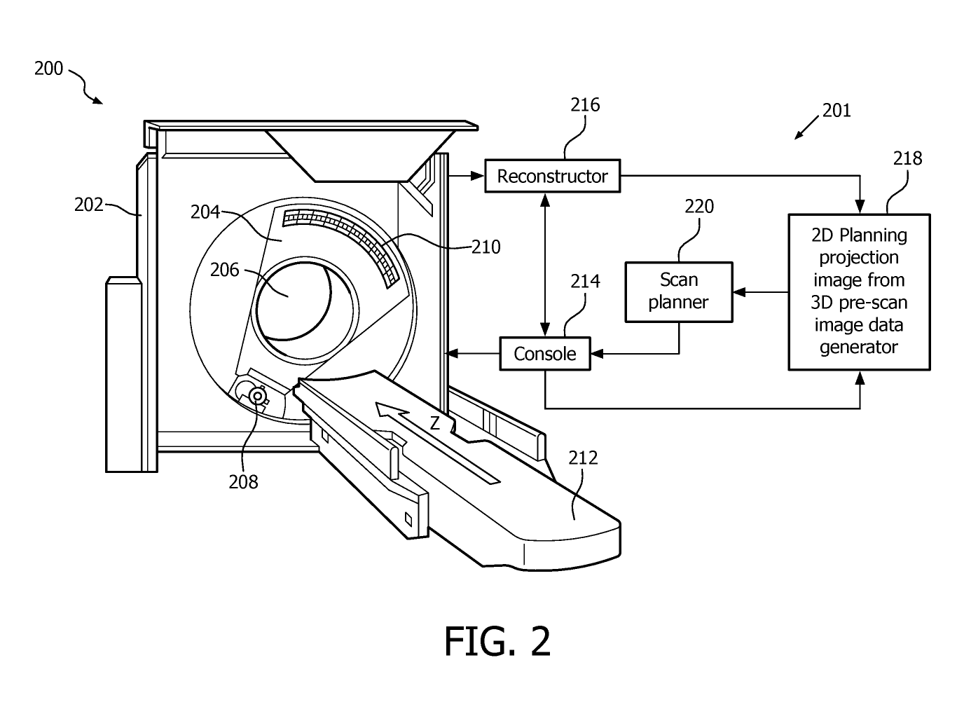

FIG. 2 schematically illustrates an example of a 2D planning projection image from 3D pre-scan image data generator in connection with an imaging system.

FIG. 3 schematically illustrates an example of lower contrast resolution 3D pre-scan image data.

FIG. 4 schematically illustrates an example of higher contrast resolution 3D pre-scan image data.

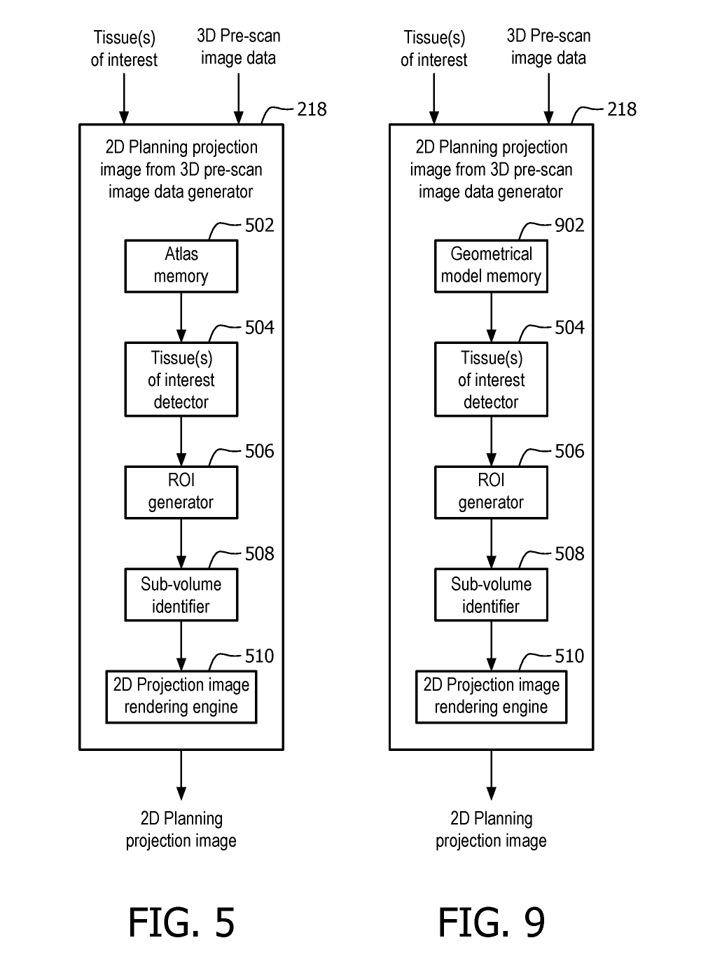

FIG. 5 schematically illustrates an example of the 2D planning projection image from 3D pre-scan image data generator that employs an anatomical atlas.

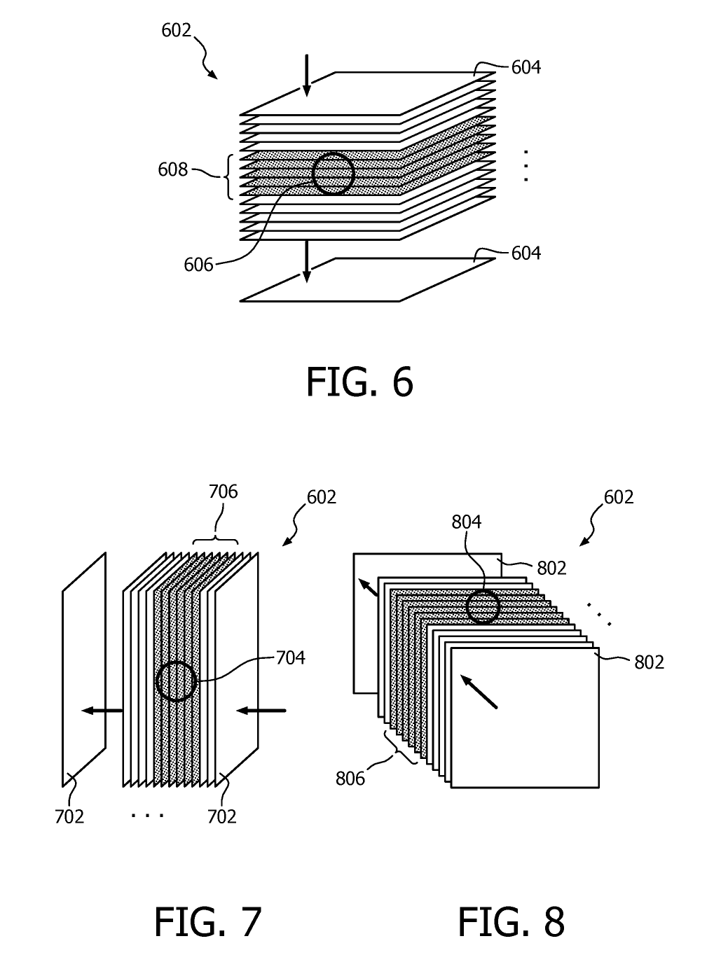

FIG. 6 schematically illustrates selection of a sub-volume of the 3D pre-scan image data corresponding to tissue of interest from a volume reformatted in a first view direction.

FIG. 7 schematically illustrates selection of the sub-volume of the 3D pre-scan image data corresponding to the tissue of interest from the volume reformatted in a second view direction.

FIG. 8 schematically illustrates selection of the sub-volume of the 3D pre-scan image data corresponding to the tissue of interest from the volume reformatted in a third view direction.

FIG. 9 schematically illustrates an example variation of the 2D planning projection image from 3D pre-scan image data generator that employs a geometrical model.

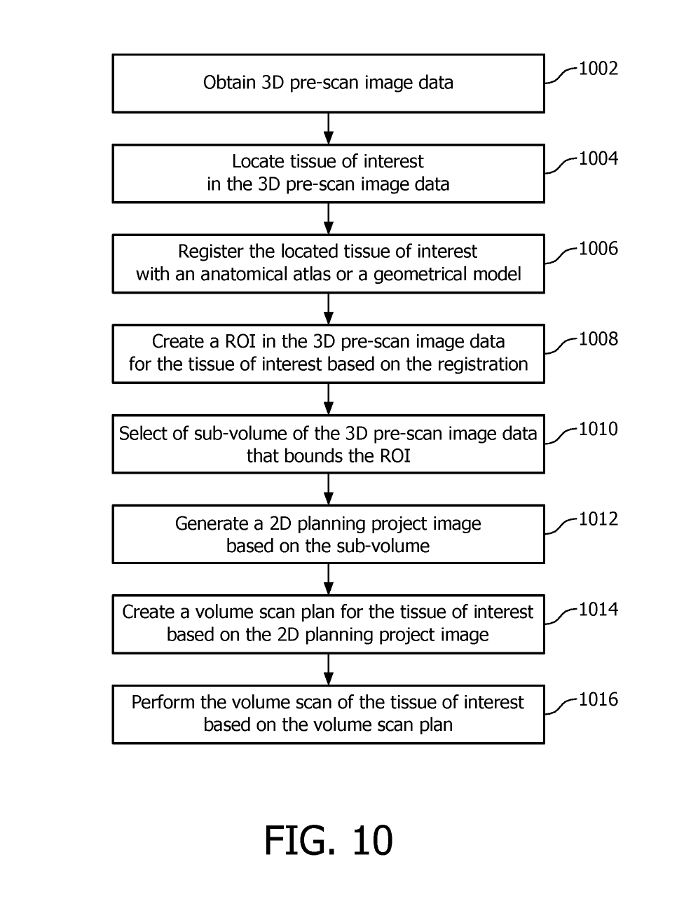

FIG. 10 illustrates an example method for generating a 2D planning project image from 3D pre-scan image data.

FIG. 2 illustrates a system 201 including an imaging system 200, such as a computed tomography (CT) scanner. The illustrated imaging system 200 includes a stationary gantry 202 and a rotating gantry 204, which is rotatably supported by the stationary gantry 202. The rotating gantry 204 rotates around an examination region 206 about a longitudinal or z-axis. A radiation source 208, such as an x-ray tube, is supported by the rotating gantry 204 and rotates with the rotating gantry 204 about the examination region 206, and emits radiation that traverses the examination region 206.

A radiation sensitive detector array 210 is located opposite the radiation source 208 across the examination region 206. The radiation sensitive detector array 210 detects radiation traversing the examination region 206 and generates a signal indicative thereof. A support 212 supports an object or subject in the examination region 206. A computer serves as an operator console 214 and includes an output device such as a display and an input device such as a keyboard, mouse, etc. Software resident on the console 214 allows the operator to control an operation of the imaging system 200 such as data acquisition.

Examples of suitable data acquisition include two-dimensional (2D) and/or three-dimensional (3D) pre-scans and include volumetric scans. An example of a 2D pre-scan is a 2D scout (also referred to as pilot or surview) scan. Generally, this type of pre-scan is a 2D projection image, similar to an x-ray. An example of a 3D pre-scan is a lower dose volumetric scan, which, generally, is not used for diagnostic purposes due to the lower image quality (e.g., lower contrast resolution). An example of lower dose image data is shown in FIG. 3. FIG. 4 shows a diagnostic image data with higher contrast resolution and covering the same field of view for image quality comparison.

An example of the volumetric scan is a helical or spiral scan with scan setting (e.g., electrical current and voltage, pitch, slice thickness, etc.), which result in an image quality at which the image data can be used for diagnostic purposes. Again, FIG. 4 shows an example of such image data. Another example of the volumetric scan is a perfusion scan in which the radiation source 208 and the scanned object/subject remain at a constant location with respect to each other and a scan of the same volume of the object or subject is repeatedly scanned over multiple revolutions or rotations of the rotating gantry 204.

Returning to FIG. 2, a reconstructor 216 reconstructs the signal generated by the radiation sensitive detector array. For example, the reconstructor 216 can reconstruct a pre-scan image data for a pre-scan scan or data acquisition and volumetric image data for a volumetric scan or data acquisition. The pre-scan image data can be a 2D projection and/or 3D lower dose image data, as discussed herein. The reconstructor 216 employs corresponding algorithms for reconstructing 2D projections, 3D pre-scan image data, volumetric image data, and/or other reconstruction algorithms.

A 2D planning projection image from 3D pre-scan image data generator 218 generates one or more 2D planning projection images from the 3D pre-scan image data. As described in greater detail below, in one instance this includes locating tissue(s) of interest in the volume of the 3D pre-scan image data, selecting a sub-volume of the 3D pre-scan image data that includes the located tissue(s) of interest, and generating the one or more 2D planning projection images based on the sub-volume. Using the sub-volume instead of the entire volume may remove structure in front of and/or behind the tissue of interest in the chosen view direction, which would otherwise visually obscure the tissue of interest in the 2D planning projection images. Using the sub-volume instead of the entire volume may also reduce planning time as there are less image slices to scroll through.

A scan planner 220 plans, with or without user interaction, a volumetric scan based the one or more 2D planning projection images. In one instance, this includes visually displaying the one or more 2D planning projection images and allowing a user to create a volume scan bounding box, which identifies at least a start position of the volumetric scan and a stop location or a length of the volumetric scan, which can be used to derive a stop location. The start and end locations define a field of view (or an extent at least along the z-axis). The field of view represents the sub-portion of the object or subject that will be scanned during the volumetric scan.

In another instance, the bounding box is automatically created and presented superimposed over one or more 2D planning projection images. In this instance, the clinician can accept, reject and/or modify the bounding box. In either instance, the one or more 2D planning projection images can be displayed with pre-set and/or optimized window/level (contrast/brightness) settings. For instance, since the thickness of the sub-volume is known and the intensity of each voxel in the sub-volume is known, an average Hounsfield unit (HU) can be computed along each of a plurality of rays through the volume, and the level can be automatically set (and accepted, rejected or modified by authorized personnel) based on the average HU value. This can be considered normalizing the intensity based on the depth of the sub-volume.

The 2D planning projection image from 3D pre-scan image data generator 218 and/or the volume scan planner 220 can be implemented via one or more computer processors (e.g., a central processing unit (CPU), a microprocessor, a controller, etc.) executing one or more computer executable instructions embedded or encoded on computer readable storage medium, which excludes transitory medium, such as physical memory. However, at least one of the computer executable instructions can alternatively be carried by a carrier wave, signal, and other transitory medium and implemented via the one or more computer processors.

The volume scan plan is provided to the console 214, which controls data acquisition based on the volume scan plan.

FIG. 5 schematically illustrates an example of the 2D planning projection image from 3D pre-scan image data determiner 218 (FIG. 2).

The 2D planning projection image from 3D pre-scan image data determiner 218 receives, as an input, 3D pre-scan image data. The 3D pre-scan image data can be from the imaging system 200 (FIG. 2), other imaging system, and/or other device. An example of another device includes, but is not limited to, a data repository such as a picture archiving and communication system (PACS), a radiology information system (RIS), an electronic medical record (EMR), a database, a server, and/or other data repository.

The 2D planning projection image from 3D pre-scan image data determiner 218 also receives, as an input, a signal indicating one or more tissues of interest. The signal can be from the console 214 (FIG. 2), a computing system implementing 2D planning projection image from 3D pre-scan image data determiner 218, the 3D pre-scan image data file (e.g., a field in the header of the file), the 3D pre-scan image data (e.g., derived from the anatomical region scanned), and/or other device.

An atlas memory 502 stores one or more anatomical atlases of one or more tissues of interest. Examples of tissues of interest include an organ such as the heart, the kidneys, etc., an anatomical region such as the chest, the pelvis, the head, etc., and/or other tissue of interest.

A tissue(s) of interest detector 504 obtains one or more anatomical atlases from the atlas memory 502 based on the signal indicating the one or more tissues of interest. The tissue(s) of interest detector 504 detects the one or more tissues of interest in the 3D pre-scan image data and registers obtained one or more anatomical atlases to the corresponding detected one or more tissues of interest in the 3D pre-scan image data. The tissue(s) of interest detector 504 can employ elastic and/or rigid registration algorithms.

In one non-limiting example, the tissue(s) of interest detector 504 detects the one or more tissues of interest in the 3D pre-scan image data and registers obtained one or more anatomical atlases to the detected one or more tissues of interest based on the approach in application Ser. No. 61/773,429, filed on Mar. 6, 2013, and entitled “Scan region determining apparatus,” the entirety of which is incorporated by reference herein.

A region of interest (ROI) generator 506 generates one or more regions of interest (ROI's) in the 3D pre-scan image data for each of the registered one or more anatomical atlases. FIG. 6 shows an example of 3D pre-scan image data 602 consisting of a plurality of slices 604 in a first view direction. FIG. 6 also shows a ROI 606 generated in 3D pre-scan image data 602 corresponding to a registration between a detected tissue of interest and an anatomical atlas.

Examples of view directions include, but are not limited to, coronal, axial, sagittal, oblique, etc. Note that the shape of the ROI 606 is provided for explanatory purposes and is not limiting, and that square, rectangular, irregular, and/or other shapes are contemplated herein. Furthermore, the region of interest (ROI) generator 506 can generate one or more other ROI's for one or more other tissues of interest in the same and/or other view direction.

FIG. 7 shows the 3D pre-scan image data 602 reformatted in a second view direction, which is orthogonal to the first view direction. In FIG. 7, the 3D pre-scan image data 602 consists of a plurality of slices 702. FIG. 7 also shows an ROI 704 generated in 3D pre-scan image data 602 corresponding to a registration between a detected tissue of interest and an anatomical atlas. Likewise, one or more other ROI's for one or more other tissues of interest can be generated in the image data 602.

FIG. 8 shows the 3D pre-scan image data 602 reformatted in a third view direction, which is orthogonal to the first and the second view directions. In FIG. 8, the 3D pre-scan image data 602 consists of a plurality of slices 802. FIG. 8 also shows an ROI 804 generated in 3D pre-scan image data 602 corresponding to a registration between a detected tissue of interest and an anatomical atlas. Similarly, one or more other ROI's for one or more other tissues of interest can be generated in the image data 602.

Returning to FIG. 5, a sub-volume of identifier 508 identifies a sub-volume of the 3D pre-scan image data that includes the one or more tissues of interest based on one or more of the ROIs 606, 704 and 804. In one non-limiting example, the sub-volume of data identifier 508 identifies the sub-volume based on the approach described in application Ser. No. 13/499,978, filed on Sep. 28, 20120, and entitled “Interactive selection of a region of interest in an image,” the entirety of which is incorporated by reference herein.

By way of non-limiting example, in FIG. 6, the sub-volume of identifier 508 identifies a sub-volume 608, which is the sub-volume that bounds the ROI 606 and, hence, the tissue of interest corresponding to the ROI 606. In FIG. 7, the sub-volume of identifier 508 identifies a sub-volume 706, which is the sub-volume that bounds the ROI 704 and, hence, the tissue of interest corresponding to the ROI 704. In FIG. 8, the sub-volume of identifier 508 identifies a sub-volume 806, which is the sub-volume that bounds the ROI 804 and, hence, the tissue of interest corresponding to the ROI 804.

Returning to FIG. 5, a 2D projection image rendering engine 510 receive one or more of the identified sub-volumes 608, 706 or 806 and generates a 2D planning projection image based thereon. In one non-limiting instance, the 2D projection image rendering engine 510 employs a digitally reconstructed radiograph (DRR) algorithm to generate the 2D planning projection image. An example DRR algorithm casts rays through the sub-volume and onto a 2D plane, and the intensity values of the voxels through which the ray traverses are combined to produce a pixel intensity value. In another non-limiting instance, another volume rendering approach can be used. For example, the 2D projection image rendering engine 510 employs a maximum intensity projection (MIP), minimum intensity projection (mIP), and/or other volume rendering technique to generate the 2D planning projection image.

The output of the 2D projection image rendering engine 510 is a 2D projection image, which, in the illustrated embodiment, represents a 2D planning projection image. By processing the sub-volume to generate the 2D projection image rather than the entire 3D pre-scan image data volume, sub-portions of the 3D pre-scan image data volume that do not include a tissue of interest and/or visually obscure tissue of interest (e.g., the perimeter of a tissue of interest) are not used to generate the 2D projection image. As a result, the 2D planning projection image may have improved image quality with respect to the tissue of interest and/or allow for more accurate and/or optimal planning of a volume scan.

For example, it may be easier to visually identify the perimeter of tissue of interest and/or a boundary between the tissue of interest and other tissue. This may allow the user to define the bounding box to ensure the entire tissue of interest (or the entirety of a sub-portion of interest of the tissue of interest) is scanned, while mitigating irradiating and dosing tissue outside of the tissue of interest. This may include tissue in a margin defined around the tissue of interest in a configuration in which the entire 3D pre-scan image data is used to generate the 2D planning projection image and structure in front of and/or behind the tissue of interest visually obscures the tissue of interest in the 2D planning projection image.

For example, for a cardiac scan, a sub-portion of the 3D pre-scan image data that includes voxels that represent the rib cage and none of the heart are excluded from or not included in the sub-volume. In this instance, this may include extracting the sub-volume and discarding the remaining volume such that the sub-volume is an actual smaller volume of data. In another instance, the voxels representing the rib cage are either visually masked, set to an intensity value of the background, and/or given a window/level and/or opacity setting that renders them visually translucent. Other approaches are also contemplated herein. Furthermore, scrolling through the sub-volume to find an image slice to plan from may consume less time relative to scrolling through the entire volume.

In FIG. 5, the 2D planning projection image from 3D pre-scan image data generator 218 registers an anatomical atlas of the tissue of interest with the 3D pre-scan image data. It is to be understood that other approaches can be used to identify the geometrical boundaries of the tissue of interest in the 3D pre-scan image data. For example, and as shown in FIG. 9, the 2D planning projection image from 3D pre-scan image data generator 218 utilizes a geometrical model from a geometrical model memory 902. The geometrical model may be a mesh based or other geometrical model. Another approach includes a manual and/or a semi-automatic approach in which the tissue of interest is outlined using a free hand drawing tool, a predetermined shape tool and/or a seed growing algorithm. Other approaches include cascaded classifiers, random decision trees, simple box detection, using intensity thresholds, etc. Still other approaches can be used to identify the geometrical boundaries of the tissue of interest.

FIG. 10 illustrates an example method for generating a 2D planning project image from 3D pre-scan image data.

It is to be appreciated that the ordering of the acts of these methods is not limiting. As such, other orderings are contemplated herein. In addition, one or more acts may be omitted and/or one or more additional acts may be included.

At 1002, obtain 3D pre-scan image data that includes voxels representing at least one tissue of interest. This may include performing a 3D pre-scan, which includes scanning the at least one tissue of interest, to generate the 3D pre-scan image data or obtaining 3D pre-scan image data from a data repository.

At 1004, the tissue of interest is located in the 3D pre-scan image data.

At 1006, the located 3D pre-scan image data is registered with an anatomical atlas or a geometric model.

At 1008, an ROI is created for the tissue of interest in the 3D pre-scan image data. As described herein, one or more ROI's can be created in one or more different reformatted view directions.

At 1010, a sub-volume of the 3D pre-scan image data that bounds or includes the tissue of interest is selected.

At 1012, a 2D planning projection image is generated based on the sub-volume. As disclosed herein, a volume rendering or other approach can be employed.

At 1014, a volume scan of the tissue of interest is created using the 2D planning projection image.

At 1016, the volume scan of the tissue of interest is performed based on the volume scan plan.

The above acts may be implemented by way of computer readable instructions, encoded or embedded on computer readable storage medium, which, when executed by a computer processor cause the processor to carry out the described acts. Additionally or alternatively, at least one of the computer readable instructions is carried by a signal, carrier wave and other transitory medium and implemented by the computer processor.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A method, comprising:
obtaining 3D pre-scan image data generated from a scan of a subject, wherein the 3D pre-scan image data includes voxels that represent a tissue of interest;
generating a 2D planning projection image showing the tissue of interest based on the 3D pre-scan image data.

2. The method of claim 1, further comprising:
creating a volume scan plan for the tissue of interest based on the 2D planning projection image, wherein the volume scan plan includes a bounding box identifying at least a start scan position for the subject.

3. The method of claim 2, further comprising:
controlling an imaging system to scan the subject based on the volume scan plan.

4. The method of claim 1, further comprising:
locating the tissue of interest in the 3D pre-scan image data;
registering the located tissue of interest with an anatomical atlas or a geometrical model; and
creating a first region of interest in the 3D pre-scan image data for the tissue of interest based on the registration,
wherein the 2D planning projection image is generated based on the first region of interest.

5. The method of claim 4, further comprising:
selecting a sub-volume of the 3D pre-scan image data corresponding to the region of interest, wherein the 2D planning projection image is generated based on the sub-volume.

6. The method of claim 5, further comprising:
using a volume rendering algorithm to generate the 2D planning projection image.

7. The method of claim 4, wherein the sub-volume includes voxels only representing the tissue of interest and does not include voxels not representing the tissue of interest.

8. The method of claim 4, wherein the first region of interest is created with the 3D pre-scan image data reformatted in a first view direction, and further comprising:
reformatting the 3D pre-scan image data in a second view direction, which is different from the first view direction; and
creating a second region of interest in the 3D pre-scan image data reformatted in the second view direction for the tissue of interest based on the registration,
wherein the 2D planning projection image is generated based on the first and the second region of interests.

9. The method of claim 8, further comprising:
reformatting the 3D pre-scan image data in a third view direction, which is different from the first and the second view direction; and
creating a third region of interest in the 3D pre-scan image data reformatted in the third view direction for the tissue of interest based on the registration,
wherein the 2D planning projection image is generated based on the first, the second, and the third region of interests.

10. The method of claim 9, wherein the 2D planning projection image is a single projection image.

11. The method of claim 10, wherein the 2D planning projection image includes a sub-projection image, one for each of the different view directions.

12. The method of claim 5, further comprising:
normalizing an intensity of the 2D planning projection image based on a thickness of the sub-volume.

13. A system, comprising:
a 2D planning projection image from 3D pre-scan image data generator that obtains 3D pre-scan image data generated from a scan of a subject, wherein the 3D pre-scan image data includes voxels that represent a tissue of interest, and generates a 2D planning projection image showing the tissue of interest based on the 3D pre-scan image data.

14. The system of claim 13, further comprising:
a scan planner that creates a volume scan plan for the tissue of interest based on the 2D planning projection image, wherein the volume scan plan includes a bounding box identifying at least a start scan position for the subject; and
an imaging system with a console that controls the imaging system to scan the subject based on the volume scan plan.

15. The system of claim 13, wherein the 2D planning projection image from 3D pre-scan image data generator comprises:
at least one of an atlas memory that stores an anatomical atlas of the tissue of interest or a geometrical model memory that stores a geometrical model of the tissue of interest;
a tissue(s) of interest detector that locates the tissue of interest in the 3D pre-scan image data; and
a region of interest generator that generates a first region of interest in the 3D pre-scan image data for the tissue of interest; and
a 2D projection image rendering engine that generates the 2D planning projection image based on the first region of interest.

16. The system of claim 15, further comprising:
a sub-volume identifier that selects a sub-volume of the 3D pre-scan image data corresponding to the region of interest, wherein the 2D projection image rendering engine generates the 2D planning projection image based on the sub-volume.

17. The system of claim 16, wherein the 2D projection image rendering engine generates the 2D planning projection image with a volume rendering algorithm.

18. The method of claim 14, wherein the sub-volume includes voxels only representing the tissue of interest and does not include voxels not representing the tissue of interest.

19. The method of claim 16, wherein the region of interest generator generates the first region of interest in a first view direction and generates at least a second region of interest in a second view direction, and the 2D projection image rendering engine generates at least one of a single image based on the first and the at least a second view directions or a different image for each of the different view directions. (Original) A computer readable storage medium encoded on computer with readable storage instructions, which, when executed by a processor of a computing system, causes the processor to:
obtain 3D pre-scan image data generated from a scan of a subject, wherein the 3D pre-scan image data includes voxels that represent a tissue of interest;
detect the tissue of interest in the 3D pre-scan image data;
generate at least one region of interest in the 3D pre-scan image;
select a sub-volume of the 3D pre-scan image data based on the at least one region of interest, wherein the sub-volume bounds the region of interest;
generate at least one 2D planning project image for the tissue of interest based on the sub-volume of the 3D pre-scan and a view direction;
plan a volume scan for the tissue of interest based on the 2D planning project image; and
perform a scan of the subject based on the volume scan.
